(19) United States
(12) Patent Application Publication
(10) Pub. No.: US 2026/0103709 A1
(43) Pub. Date: Apr. 16, 2026

(54) C-MYC-TARGETING CHARGE-ALTERING RELEASABLE TRANSPORTERS AS ANTI-TUMOR AGENTS FOR BREAST CANCER THERAPY

(71) Applicant: The Board of Trustees of the Leland Stanford Junior University, Stanford, CA (US)

(72) Inventors: Dean W. Felsher, Stanford, CA (US); Robert M. Waymouth, Stanford, CA (US); George W. Sledge, Jr., Stanford, CA (US); Wadie D. Mahauad-Fernandez, Stanford, CA (US)

(73) Assignee: The Board of Trustees of the Leland Stanford Junior University, Stanford, CA (US)

(21) Appl. No.: 19/116,122

(22) PCT Filed: Sep. 29, 2023
(86) PCT No.: PCT/US23/34106

§ 371 Date: Mar. 27, 2025

Related U.S. Application Data
(60) Provisional application No. 63/377,555, filed on Sep. 29, 2022.

(51) Int. Cl. C12N 15/113; A61K 39/00; A61K 45/06; A61P 35/00; C07K 16/30

(52) U.S. Cl. CPC C12N 15/1135; A61K 45/06; A61P 35/00; C07K 16/30; A61K 2039/54; C12N 2310/14; C12N 2310/3515

(57) ABSTRACT

There are provided herein, inter alia, a copolymer, cell penetrating complexes, compositions and methods for the delivery of therapeutic, including small interfering RNA-based therapeutic agents, into a cell and related methods for treating cancer, including breast cancer and triple-negative breast cancer.

### BACKGROUND

Genetic inactivation of c-Myc has been shown to produce rapid tumor regression and re-establishment of anti-cancer immunity in several model systems. However, due to its nuclear localization and lack of defined enzymatic activity, Myc has been described as “undruggable” and pharmacological-mediated Myc inhibition has proven difficult. Instead of targeting Myc directly, various approaches have targeted its transcriptional regulators, its heterodimerization with MAX, its DNA-binding capability, or its phosphorylation to promote degradation, all with limited success.

Another approach is the use of small interfering RNA molecules (siRNAs) which can inhibit expression of a target gene. A Dicer-substrate small interfering RNA targeting MYC (DCR-MYC) formulated in lipid nanoparticles achieved early success in Phase I studies against various solid tumors. Tolcher et al., J Clin. Oncol. 33 15 Suppl Meeting Abstracts of 2015 ASCO Meeting, Abstract #11006. However, siRNA therapeutics, including DCR-MYC, have faced obstacles to their effective use in treating cancer, including chemical instability, inefficient delivery systems, and lack of tumor specificity resulting in unacceptable toxicity.

The present invention addresses the need for improved compositions and methods for efficient delivery of siRNAs to tumors, particularly an anti-MYC siRNA for use in the treatment of cancer.

### SUMMARY

The present invention provides compositions and methods for cancer therapy targeted to c-MYC expressing tumors. In one aspect, the invention provides compositions including a c-Myc directed small interference RNA (siRNA) non-covalently attached to a co-oligomer of cationic alpha aminoester and lipophilic monomer repeating units. In embodiments, the lipophilic monomer is a mixed lipophilic monomer.

In one aspect, the invention provides compositions including a c-Myc directed small interference RNA (siRNA) non-covalently attached to a co-oligomer of cationic alpha aminoester and lipophilic monomer repeating units wherein the lipophilic monomer is a mixed lipophilic monomer comprising oleyl and nonenyl lipid moieties having the structure of Compound 1.

In one aspect, the invention provides compositions including a c-Myc directed small interference RNA (siRNA) non-covalently attached to a co-oligomer having the structure of Compound 3.

In one aspect, the invention provides compositions including a c-Myc directed small interference RNA (siRNA) non-covalently attached to a co-oligomer having the structure of Compound 4.

The composition may also include where the siRNA has a sequence comprising SEQ ID NO: 1, or a sequence having at least 80% identity to SEQ ID NO: 1.

The composition may also include a plurality of tumor-targeted antibodies conjugated to the copolymer. In embodiments, the tumor-targeted antibodies are anti-TROP2 antibodies.

The composition may also include one or more imaging agents or radiotherapeutic agents, either conjugated to the copolymer or, in the case of isotopes, incorporated within the structure of the copolymer. In embodiments, the one or more imaging agents is selected from a suitable metal ion or isotope, for example, fluorine, lutetium, actinium, gallium, copper, samarium, radium, yttrium, palladium, iridium, gadolinium or lead. In embodiments, the one or more imaging agents includes a fluorophore selected from cyanine, crystal violet, eosin, fluorescein, malachite green, Oregon green, rhodamine, and Texas Red. In embodiments, the radioimaging or radiotherapeutic agent is selected from radium-233, lutetium-177, technetium-99, and yttrium-90. Other technical features may be readily apparent to one skilled in the art from the following figures, descriptions, and claims.

The invention also provides pharmaceutical compositions comprising a c-Myc directed small interference RNA (siRNA) non-covalently attached to a co-oligomer having the structure of a compound of Formula I, and a pharmaceutically acceptable carrier. In embodiments, the co-oligomer is selected from the group consisting of Compound 1, Compound 2, Compound 3, Compound 4, Compound 5, Compound 6, and Compound 7. In embodiments, the co-oligomer is selected from the group consisting of Compound 1, Compound 3, and Compound 4. In embodiments, the pharmaceutical composition comprises a mixture of two or more co-oligomers as described herein. In embodiments, the pharmaceutical composition comprises a mixture of Compound 1 and any one of Compound 2, Compound 3, Compound 4, Compound 5, Compound 6, or Compound 7. In embodiments, the pharmaceutical composition comprises a mixture of Compound 6 and Compound 7. In embodiments, the pharmaceutical composition is formulated as a liquid suitable for injection. Other technical features may be readily apparent to one skilled in the art from the following figures, descriptions, and claims.

The invention also provides methods of treating cancer in a subject in need thereof, the methods comprising administering a pharmaceutical composition comprising a c-Myc directed small interference RNA (siRNA) non-covalently attached to a compound of Formula I. In embodiments, provided are methods of treating cancer in a subject in need thereof, the methods comprising administering a pharmaceutical composition comprising a c-Myc directed small interference RNA (siRNA) non-covalently attached to a co-oligomer selected from the group consisting of Compound 1, Compound 2, Compound 3, Compound 4, Compound 5, Compound 6, and Compound 7. In embodiments, the co-oligomer is selected from the group consisting of Compound 1, Compound 3, and Compound 4. In embodiments, the pharmaceutical composition comprises a mixture of two or more co-oligomer as described herein. In embodiments, the pharmaceutical composition comprises a mixture of Compound 1 and any one of Compound 2, Compound 3, Compound 4, Compound 5, Compound 6, or Compound 7. In embodiments, the pharmaceutical composition comprises a mixture of Compound 6 and Compound 7.

In embodiments, the cancer is one whose cells overexpress c-Myc compared to the expression of c-Myc in non-cancerous tissue. In embodiments c-Myc is considered to be overexpressed where its expression is elevated at least 2-fold relative to its expression in a reference non-cancerous tissue. In embodiments, the cancer is selected from the group consisting of breast cancer, liver cancer, kidney cancer, lung cancer, ovarian cancer, and bone cancer. In embodiments, the cancer is a leukemia or lymphoma. In embodiments, the cancer is refractory to standard therapy. In embodiments, the cancer is recurrent. In embodiments, the cancer is unresectable locally advanced or metastatic.

In embodiments, the cancer is breast cancer. In embodiments, the breast cancer is refractory to standard therapy. In embodiments, the breast cancer is recurrent. In embodiments, the breast cancer is unresectable locally advanced or metastatic. In embodiments, the breast cancer is characterized as lacking one or more of estrogen receptor, progesterone receptor, and human epidermal growth factor receptor 2 (HER2). In embodiments, the breast cancer is characterized as lacking all three of estrogen receptor, progesterone receptor, and human epidermal growth factor receptor 2 (HER2).

In embodiments, the method further includes administering an immunotherapy agent to the subject. In embodiments, the immunotherapy agent is a programmed cell death protein 1 (PD-1) or programmed cell death ligand-1 (PD-L1) inhibitor. In embodiments, the immunotherapy agent is selected from the group consisting of pembrolizumab, nivolumab, cemiplimab, dostarlimab, atezolizumab, avelumab, and durvalumab.

In embodiments, the composition is administered by an intravenous route.

In embodiments, administration is by intratumoral injection.

The invention also provides methods of eliciting an anti-tumor immune response in a subject having cancer, the method comprising administering to the subject a pharmaceutical composition comprising a c-Myc directed small interference RNA (siRNA) non-covalently attached to a co-oligomer having the structure of Formula I. In embodiments, provided are methods of eliciting an anti-tumor immune response in a subject having cancer, the methods comprising administering a pharmaceutical composition comprising a c-Myc directed small interference RNA (siRNA) non-covalently attached to a co-oligomer selected from the group consisting of Compound 1, Compound 2, Compound 3, Compound 4, Compound 5, Compound 6, and Compound 7. In embodiments, the co-oligomer is selected from the group consisting of Compound 1, Compound 3, and Compound 4. In embodiments, the pharmaceutical composition comprises a mixture of two or more co-oligomer as described herein. In embodiments, the pharmaceutical composition comprises a mixture of Compound 1 and any one of Compound 2, Compound 3, Compound 4, Compound 5, Compound 6, or Compound 7. In embodiments, the pharmaceutical composition comprises a mixture of Compound 6 and Compound 7.

The invention also provides methods for clinical imaging of a c-Myc-expressing tumor in a subject, the method includes administering to the subject an imaging composition comprising a c-Myc directed small interference RNA (siRNA) non-covalently attached to a co-oligomer having the structure of Formula I, wherein the co-oligomer is conjugated to an imaging agent, or wherein the co-oligomer incorporates the imaging agent. In embodiments, provided are methods for clinical imaging of a c-Myc-expressing tumor in a subject, the methods comprising administering an imaging composition comprising a c-Myc directed small interference RNA (siRNA) non-covalently attached to a co-oligomer selected from the group consisting of Compound 1, Compound 2, Compound 3, Compound 4, Compound 5, Compound 6, and Compound 7, wherein the co-oligomer is conjugated to an imaging agent, or wherein the co-oligomer incorporates the imaging agent. In embodiments, the co-oligomer is selected from the group consisting of Compound 1, Compound 3, and Compound 4. In embodiments, the imaging composition comprises a mixture of two or more co-oligomer as described herein. In embodiments, the imaging composition comprises a mixture of Compound 1 and any one of Compound 2, Compound 3, Compound 4, Compound 5, Compound 6, or Compound 7. In embodiments, the imaging composition comprises a mixture of Compound 6 and Compound 7, wherein the copolymer is conjugated to an imaging agent, or wherein the co-oligomer incorporates the imaging agent.

The invention also provides methods of treating a solid tumor in a subject in need thereof, the methods comprising administering to the subject a composition comprising a c-Myc directed small interference RNA (siRNA) non-covalently attached to a co-oligomer having the structure of Formula I, or having the structure of a co-oligomer selected from the group consisting of Compound 1, Compound 2, Compound 3, Compound 4, Compound 5, Compound 6, and Compound 7, where the tumor cells are characterized as over-expressing c-Myc or having high levels of c-Myc activity compared to reference non-cancerous tissue. In embodiments, the co-oligomer is selected from the group consisting of Compound 1, Compound 3, and Compound 4. Other technical features may be readily apparent to one skilled in the art from the following figures, descriptions, and claims.

### BRIEF DESCRIPTION OF THE DRAWINGS

FIG. 1A to FIG. 1D: Evaluation of different anti-MYC siRNAs and different CART vehicles in 4T1 TNBC cells. Relative mRNA levels of Myc (FIG. 1A) and the Myc target genes Apex1 (FIG. 1B) and Gnl3 (FIG. 1C)₂₄ or 48 hours after transfection of 4T1 cells with lipofectamine alone (no siRNA) or with each of five different Myc-targeting siRNAs (siMYC 1-5). siMYC 1 and siMYC 2 were the most effective siRNAs. All samples were normalized using UBC as the house keeping gene and the “siGLO” sample set to one. (FIG. 1D) Fluorescent-based binding assay to identify the CART with the most affinity for 4T1 TNBC cells. 4T1 cells were treated for 2 or 4 hours with either the fluorescent molecule siGLO alone, or each of eight different CARTs loaded with siGLO, or lipofectamine loaded with siGLO. Error bars represent SEM. Significance was taken at P<0.01 (**) and P<0.005 (***). ns=not significant.

FIG. 2A to FIG. 2B: Myc mRNA expression in TNBC cells treated with CART 1 siMYC 2, CART 3 siMYC 2 and lipofectamine loaded with siMYC 2 (FIG. 2A). mRNA levels of the Myc target Apex1 under the same conditions (FIG. 2B).

FIG. 3A to FIG. 3B: Viability of several murine and human TNBC cells following JQ1 treatment. Relative cell viability and IC50 values of (FIG. 3A) the murine cell lines M1011, M158, and 4T1; and, (FIG. 3B) the human cell lines MDA-MB-436, MDA-MB-231, and BT20 following treatment with JQ1 at different concentrations ranging from 0-100 μM. for 24 hours.

FIG. 4A to FIG. 4F: CART siMYC decreases cell viability in cells expressing high levels of Myc. Cell viability of mouse (FIG. 4A) M158 cells, (FIG. 4B) M1011 cells, and (FIG. 4C)₄T1 cells treated with the Myc-targeting BETi JQ1 at different concentrations ranging from 0-1 μM for two hours following by treatment with either CART alone, CART siLUC (targeting Luciferase), or CART siMYC (targeting mouse Myc) for 48 hours. Cell viability of human (FIG. 4D) MDA-MB-231 cells, (FIG. 4E) MDA-MB-436 cells, and (FIG. 4F) BT20 cells treated with the MYC-targeting drug JQ1 at different concentrations ranging from 0-1 μM for two hours following by treatment with either CART alone, CART siLUC, or CART siMYC (targeting human MYC) for 48 hours. Error bars represent SEM. Significance was taken at P<0.05 (*), P<0.01 (**), and P<0.005 (***). ns=not significant.

FIG. 5A to FIG. 5E: Intratumoral or intravenous delivery of CART siMYC effectively decreases TNBC tumor growth. FIG. 5A, body weight over time up to 6 days. FIG. 5B, representative gross TNBC tumor images. Tumor volume over time calculated at days 0, 3, 6, 9, 12, 15, and 18 days after treatment using bioluminescence imaging (FIG. 5C) or caliper measurements (FIG. 5D), and (FIG. 5F) tumor weight at the time of euthanasia. BALB/c mice bearing 4T1 TNBC tumors were treated with a CART loaded with a scramble siRNA control (siCTL) or CART loaded with a Myc-targeting siRNA (siMYC) delivered intravenously (IV), intratumorally (IT), or intraperitoneally (IP) at days 3, 6, and 9 post treatment enrollment. CART siCTL was only delivered intravenously. Scale bar in panel b=10 mm. Error bars represent SEM. 575 Significance was taken at P<0.05 (*) and P<0.01 (**). ns=not significant.

FIG. 6A to FIG. 6D: CART siMYC reduces growth of TNBC tumors. (FIG. 6A) Representative BLI images, (FIG. 6B) tumor volume over time, (FIG. 6C) gross images of TNBC tumors and (FIG. 6D) tumor weight at the time of euthanasia from BALB/c mice bearing 4T1 TNBC 580 tumors on the left and right flanks. TNBC tumors on the left flank were treated (T) with siMYC alone, CART siCTL, or CART siMYC at days 3, 6, and 9 post treatment enrollment. TNBC tumors on the right flank remained untreated (U). Scale bar in panel c=10 mm. Error bars represent SEM. Significance was taken at P<0.05 589 (*) and P<0.01 (**). ns=not significant.

FIG. 7A to FIG. 7D: CART siMYC reduces the growth rate of treated and untreated TNBC tumors in the same host. (FIG. 7A) Graphical depiction of the in vivo experiment. (FIG. 7B) Body weight, (FIG. 7C) representative BLI images, and (FIG. 7D) tumor volume over time of mice bearing two adjacent TNBC tumors and treated with triweekly doses of CART siLUC or CART siMYC until day 18 which was the experimental endpoint. Error bars represent SEM. Significance was taken at P<0.05 (*). ns=not significant.

FIG. 8A to FIG. 8E: (FIG. 8A) Representative immunohistochemistry images and quantification of cells positive for (FIG. 8B) Myc, (FIG. 8C) CD4, and (FIG. 8D) F4/80 from treated (T) and untreated (U) TNBC tumors of mice injected with CART siLUC or CART siMYC. Arrow heads depict CD4 positive cells. Scale bar=50 μm. Error bars represent SEM. Significance was taken at P<0.05 (*), P<0.01 (**), and P<0.005 (***). ns=not significant. (FIG. 8E) Relative mRNA levels of Myc, Apex1, Gnl3, and Srm from treated and untreated TNBC tumors in mice injected with CART siCTL or CART siMYC for 18 days. All samples were normalized using UBC as the house keeping gene and the “Treated Tumors CART siLUC” sample was set to one.

### DETAILED DESCRIPTION

The present invention provides compositions and methods for cancer therapy. The compositions described here are targeted against c-Myc expressing cancer. In embodiments, the compositions are particularly for use in the treatment of solid tumors, such as breast cancer, including triple-negative breast cancer. The compositions of the invention comprise a cationic polymer-based delivery vehicle complexed with an anti-Myc small interfering RNA (siRNA). The delivery vehicle comprises repeating units of a cationic alpha aminoester and lipophilic monomer, which may include a mixture of two lipid moieties. In embodiments, the mixed lipophilic monomer comprises oleyl and nonenyl lipid moieties. As demonstrated herein, the delivery vehicle is readily taken up by cancer cells and delivers its siRNA cargo into the cells where it is effective to reduce both Myc mRNA expression and protein levels as well as Myc-mediated signaling, resulting in substantial anti-tumor activity in animal model systems.

### Definitions

Unless the context indicates otherwise, it is specifically intended that the various features of the disclosure described herein can be used in any combination. Moreover, the disclosure also contemplates that in some embodiments any feature or combination of features set forth herein can be excluded or omitted. To illustrate, if the specification states that a complex has components A, B and C, it is specifically intended that any of A, B or C, or a combination thereof, can be omitted and disclaimed singularly or in any combination.

It must be noted that as used herein and in the appended claims, the singular forms “a”, “an”, and “the” include plural referents unless the context clearly dictates otherwise. Thus, for example, reference to “a cancer cell” includes a plurality of cancer cells. In other examples, reference to “a nucleic acid” or “nucleic acid” includes a plurality of nucleic acid molecules, i.e. nucleic acids.

The term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

Also as used herein, “and/or” refers to and encompasses any and all possible combinations of one or more of the associated listed items, as well as the lack of combinations when interpreted in the alternative (“or”).

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

The abbreviations used herein have their conventional meaning within the chemical and biological arts. The chemical structures and formulae set forth herein are constructed according to the standard rules of chemical valency known in the chemical sciences.

The terms “oligomer” and “polymer” are used interchangeably herein to refer to a compound that has a plurality of repeating subunits, which may be referred to as blocks or monomer units, or simply as monomers. The terms “co-oligomer” or “copolymer” are used interchangeably herein to refer to an oligomer or polymer that includes two or more different types of monomers. For example, in the context of the present invention the compounds provided are co-oligomers, which may also be referred to as copolymers, comprising at least two different types of monomers, a lipophilic block and an alpha aminoester block. Thus, the co-oligomers described herein may also be referred to herein as co-oligomers of cationic alpha aminoester and lipophilic monomer repeating units.

The term “polymerizable monomer” is used in accordance with its meaning in the art of polymer chemistry and refers to a compound that may covalently bind chemically to other monomer molecules (such as other polymerizable monomers that are the same or different) to form a polymer.

The term “block copolymer” is used in accordance with its ordinary meaning and refers to two or more portions (e.g., blocks) of polymerized monomers linked by a covalent bond. In embodiments, a block copolymer is a repeating pattern of polymers. In embodiments, the block copolymer includes two or more monomers in a periodic (e.g., repeating pattern) sequence. For example, a diblock copolymer has the formula: —B—B—B—B—B—B-A-A-A-A-A-, where ‘B’ is a first subunit and ‘A’ is a second subunit covalently bound together. A triblock copolymer therefore is a copolymer with three distinct blocks, two of which may be the same (e.g., A-A-A-A-A B—B—B—B—B—B A-A-A-A-A) or all three are different (e.g., A-A-A-A-A B—B—B—B—B—B—C—C—C—C—C—) where ‘A’ is a first subunit, ‘B’ is a second subunit, and ‘C’ is a third subunit, covalently bound together. The term “random copolymer” refers to monomers randomly linked together in the polymer chain. The terms “random copolymer” and “statistical copolymer” are used interchangeably. The copolymers described herein may be block or random copolymers. For example, a diblock lipid could be a blocked or random mixture of two lipids. Similarly, a triblock copolymer comprising two lipid blocks and a cationic block could be blocked or random with respect to the sequence of monomer residues.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃, —CH₂—O—Si(CH₃)₃, R—S—S—R′, RO—S(O)x-OR′, and RO—P(O)x-OR′. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples o]f heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O) R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzooxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

The terms “Nucleic acid” and “polynucleotide” may be used interchangeably to refer to deoxyribonucleotides or ribonucleotides and polymers thereof in either single-, double- or multiple-stranded form, or complements thereof. The terms “polynucleotide,” “oligonucleotide,” and “oligo” are also used interchangeably. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides include single and double stranded DNA, single and double stranded RNA, and hybrid molecules having mixtures of single and double stranded DNA and RNA. RNA may include messenger RNA (mRNA), small interference RNA (siRNA), short hairpin RNA (shRNA), micro RNA (miRNA), guide RNA (gRNA), CRISPR RNA (crRNA), and transactivating RNA (tracrRNA). DNA may include plasmid DNA (pDNA), minicircle DNA, genomic DNA (gNDA), and fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids has one or more arms or branches of nucleotides. Optionally, the branched nucleic acids are repetitively branched to form higher ordered structures such as dendrimers and the like.

Polynucleotides may comprise known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms apply to macrocyclic peptides, peptides that have been modified with non-peptide functionality, peptidomimetics, polyamides, and macrolactams. A “fusion protein” refers to a chimeric protein encoding two or more separate protein sequences that are recombinantly expressed as a single moiety.

The terms “peptidyl” and “peptidyl moiety” means a monovalent peptide.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Contacting” is used in accordance with its plain ordinary meaning and refers to the process of allowing at least two distinct species (e.g. chemical compounds including biomolecules or cells) to become sufficiently proximal to react, interact, associate, or physically touch. It should be appreciated, however, that the resulting reaction product can be produced directly from a reaction between the added reagents or from an intermediate from one or more of the added reagents which can be produced in the reaction mixture. In embodiments, contacting includes, for example, allowing a nucleic acid to interact with an endonuclease.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “exogenous” refers to a molecule or substance (e.g., nucleic acid or protein) that originates from outside a given cell or organism. Conversely, the term “endogenous” refers to a molecule or substance that is native to, or originates within, a given cell or organism.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, having the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetofection and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

As used herein, the terms “cationic charge altering releasable transporter,” “CART” and the like refer to the co-oligomer portion of the compositions disclosed herein. The CART compounds are able to release the siRNA component of the composition within a cell via an intramolecular rearrangement of the poly(alpha-aminoester) component of the co-oligomer, which occurs rapidly at intracellular pH, e.g., pH of about 7.4. In embodiments, the co-oligomer degrades rapidly at pH 7.4 with a half-life of less than 20 min, preferably less than 10 min or less than 5 min.

The compositions comprising copolymer and siRNA advantageously condense to form nanoparticles which serve to protect the siRNA cargo and may also further facilitate cellular entry. Such nanoparticles are generally in a size range of from 50-400 nanometers (nm) or from about 100-300 nm. In embodiments, a composition of the invention comprising an siRNA non-covalently attached to a co-oligomer as described herein forms a nanoparticle having a mean particle size (7) in the range of 100-300 nanometers (nm), a distribution size of from 100-300 nm, a polydispersity index of from 0.10-0.040, and a Zeta potential of from about 10-50 mV, preferably from about 10-30 mV.

The term “amphipathic polymer” as used herein refers to a polymer containing both hydrophilic and hydrophobic portions. In embodiments, the hydrophilic to hydrophobic portions are present in a 1 to 1 mass ratio. In embodiments, the hydrophilic to hydrophobic portions are present in a 1 to 2 mass ratio. In embodiments, the hydrophilic to hydrophobic portions are present in a 1 to 5 mass ratio. In embodiments, the hydrophilic to hydrophobic portions are present in a 2 to 1 mass ratio. In embodiments, the hydrophilic to hydrophobic portions are present in a 5 to 1 mass ratio. An amphipathic polymer may be a diblock or triblock copolymer. In embodiments, the amphiphilic polymer may include two hydrophilic portions (e.g., blocks) and one hydrophobic portion (e.g., block)

The term “initiator” refers to a compound that is involved in a reaction synthesizing a co-oligomer having the purpose of initiating the polymerization reaction. Thus, the initiator is typically incorporated at the end of a synthesized polymer. For example, a plurality of molecules of one type (or formula) of monomer or more than one type of monomers (e.g. two different types of monomers) can be reacted with an initiator to provide a co-oligomer. The initiator can be present on at least one end of the resulting polymer and not constitute a repeating (or polymerized) unit(s) present in the polymer.

### Compositions

Provided are compositions comprising a small interference RNA (siRNA) non-covalently attached to a co-oligomer of cationic alpha aminoester and lipophilic monomer repeating units. In embodiments, one or more counter ions (e.g., anions) may also be present as countercharges to the positive charges in the co-polymer. In embodiments, the ratio of cationic charge in the poly(alpha aminoester) backbone to anionic charge of the siRNA molecule is about 20:1, 10:1, or 5:1.

In some embodiments, the co-oligomers described here may be derived from cyclic amino-ester and cyclic methyl trimethylene carbonate (MTC) monomers. Cyclic amino-esters have the base structure of morpholin-2-one and homologs thereof, with multiple substitution patterns possible including N-acylation with a variety of hydrophobic groups (e.g., R=alkyl, alkenyl, aryl, polycycles including steroids, heterocycles), cationic groups (e.g., ammonium, phosphonium, sulfonium, guanidinium, including acylation with amino acids such as glycine, lysine, ornithine, arginine), anionic groups (e.g., carboxylate, sulfate, phosphate), or hydrophilic (e.g., PEG) carbamates. Protection of the morpholine nitrogen with N-Boc or N-Cbz groups followed by organocatalytic ring opening oligomerization or polymerization can afford upon deprotection cationic polymer or oligomer backbones. Other substitutions may include alpha-alkylation or functionalization next to the ester carbonyl with the aforementioned possible functionalities selected to allow for cargo complexation and subsequent cargo release by biodegradation; alkylation proximal to the morpholine nitrogen with the aforementioned functionalities; and combinations of these modifications.

Copolymers or co-oligomers (block or statistical) can also be made by mixing two or more morpholin-2-one monomers, or by the copolymerization (or co-oligomerization) of one or multiple morpholin-2-one monomers with one or multiple cyclic carbonate monomers described herein. These carbonate monomers can incorporate a similar variety of side chain functionality, notably lipophilic groups or cationic groups to modulate oligonucleotide stability, delivery, and release properties. Furthermore, a variety of other commercially available cyclic ester monomers can be used including but not limited to lactide, glycolide, valerolactone, and/or caprolactone to incorporate lipophilic functionality.

In general, the synthesis of poly(aminoester)s and poly(carbonate-co-aminoester)s is achieved through the ring-opening polymerization and/or copolymerization of morpholine-2-one and cyclic carbonate monomers. The N-Boc protected morpholinone (MBoc) polymerizes to high conversion (>85%), tunable Mn (1 kDa-20 kDa), and low molecular weight distributions (Mw/Mn-1.1-1.3) using an organocatalytic system. Post-polymerization deprotection of the Boc groups affords a cationic (diprotic, secondary amine) water-soluble polymer (−0.5 M in D20, stable for >3 days). Furthermore, copolymerization of MBoc with MTC-dodecyl carbonate monomers followed by deprotection give rise to moderately charged cationic materials in high yield (>60%) with narrow polydispersity <1.4 PDI) and tunable block length. Block length is controlled by the ratio of initiator to monomer.

Additional detailed methods of making a poly(alpha aminoester) moiety are described in Blake et al., J. Am Chem Soc. 2014 136:9252-9255 and Blake et al., Chem Sci 2020 11:2951. Detailed methods of making copolymers of poly(aminoester)s and lipophilic polymers, including mixed lipophilic polymers, are described in WO 2018/022930 and WO 2020/097614.

The poly(aminoester) component of the co-oligomers described here is biocompatible and biodegradable. In certain embodiments, the poly(aminoester) component rapidly degrades through a unique pH-dependent intramolecular rearrangement to generate degradation products that are substantially nontoxic when the co-oligomer is administered in a therapeutic amount to a subject. The carbonate segment of the poly(aminoester) component of the co-oligomer degrades through hydrolysis and decarboxylation, and its byproducts have previously been shown to be non-toxic.

In embodiments, the methods described here may include complexation of the siRNA cargo with a co-oligomer in the presence of a coordinating metal such as Zn⁺², Mg⁺², Ca⁺²; a dynamic non-covalent cross linker such as a carbohydrate; a counterion such as Cl⁻, AcO⁻, succinate, or citrate; or a solubility modulator such as a lipid or a polyethyleneglycol (PEG), or any combination thereof.

In embodiments, the siRNA comprises the nucleotide sequence of SEQ ID NO: 1 or a nucleotide sequence having at least 80%, at least 85%, at least 90%, at least 95%, or at least 99% sequence identity thereto.

In embodiments, the siRNA comprises the nucleotide sequence of SEQ ID NO: 2 or SEQ ID NO: 3, or a nucleotide sequence having at least 80%, at least 85%, at least 90%, at least 95%, or at least 99% sequence identity thereto.

TABLE 1
anti-Myc siRNA sequences

| Seq Identifier | NAME | SEQUENCE |
|---|---|---|
| SEQ ID NO: 1 | Human | CGAUGUUGUUUCUGUGGAA |
| | siMYC4 | |
| SEQ ID NO: 2 | Mouse | GAAACGACGAGAACAGUUG |
| | siMYC1 | |
| SEQ ID NO: 3 | Mouse | CCACUCACCAGCACAACUA |
| | siMYC2 | |

In embodiments, the co-oligomer has a structure of Formula I:

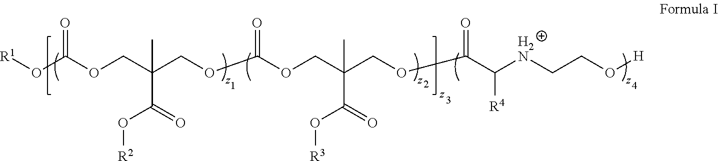

where z1 and z2 are independently from 0 to 100, and at least one of z1 or z2 is not 0;
z3 and z4 are independently from 1 to 100;
R¹ is hydrogen, halogen, fingolimod, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O) NHNH₂, —NHC(O) NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ are independently hydrogen, unbranched C₁-C₃₀ alkyl, which may be fully saturated, mono- or polyunsaturated, or cholesterol, optionally wherein R² and R³ independently hydrogen, stearyl, oleyl, linoleyl, dodecyl, nonenyl, or cholesterol; and
R⁴ is independently hydrogen, unbranched C₁-C₁₀ alkyl or heteroalkyl, —CH₂CH₂CH₂NH₃ or —CH₂CH₂CH₂CH₂NH₃.

### In an Embodiment of Formula I,

z1 and z2 are independently from 0 to 25, and at least one of z1 or z2 is not 0;
z3 and z4 are independently from 1 to 25;
R¹ is fingolimod or unsubstituted aryl;
R² and R³ are independently hydrogen, unbranched C₁-C₃₀ alkyl, which may be fully saturated, mono- or polyunsaturated, or cholesterol, optionally wherein R² and R³ independently hydrogen, stearyl, oleyl, linoleyl, dodecyl, nonenyl, or cholesterol; and
R⁴ is independently hydrogen, unbranched C₁-C₁₀ alkyl or heteroalkyl, —CH₂CH₂CH₂NH₃ or —CH₂CH₂CH₂CH₂NH₃.

### In an Embodiment of Formula I,

z1 is from 1 to 25;
z2 is 0;
z3 and z4 are independently from 1 to 25;
R¹ is fingolimod or unsubstituted aryl;
R² and R³ are independently hydrogen, unbranched C₁-C₃₀ alkyl, which may be fully saturated, mono- or polyunsaturated, or cholesterol, optionally wherein
R² and R³ independently hydrogen, stearyl, oleyl, linoleyl, dodecyl, nonenyl, or cholesterol; and
R⁴ is independently hydrogen, unbranched C₁-C₁₀ alkyl or heteroalkyl, —CH₂CH₂CH₂NH₃ or —CH₂CH₂CH₂CH₂NH₃.

In embodiments, the co-oligomer is selected from the group consisting of Compound 1, Compound 2, Compound 3, Compound 4, Compound 5, Compound 6, or Compound 7. In embodiments, the co-oligomer is a mixture of Compound 6 and Compound 7, referred to as Compound 8 in Table 2.

TABLE 2
Exemplary co-oligomers

| | |
|---|---|
| 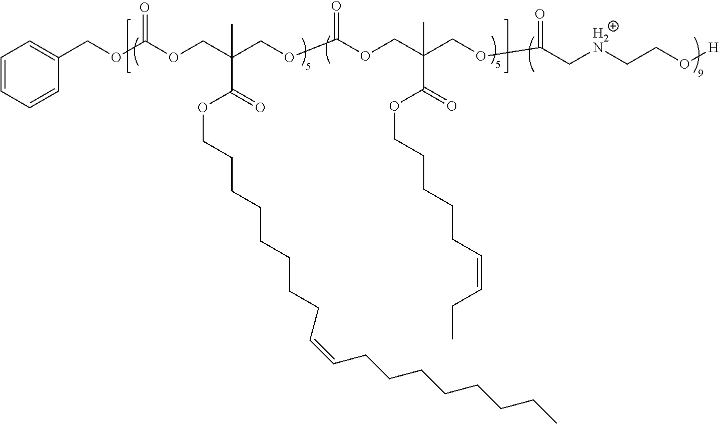 | Compound 1 |
| 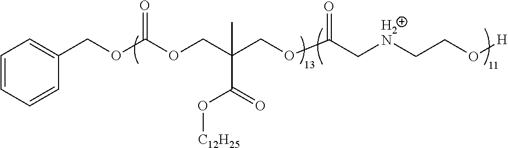 | Compound 2 |
| 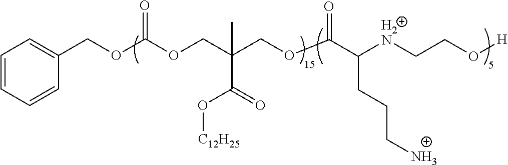 | Compound 3 |
| 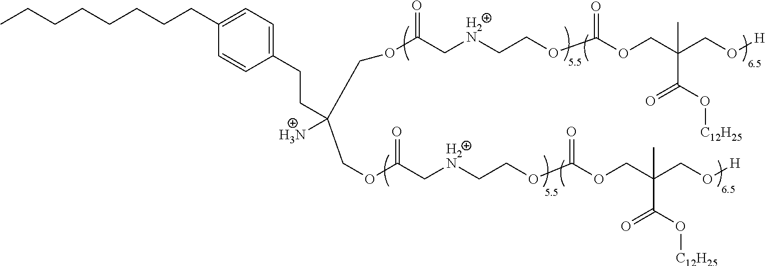 | Compound 4 |
| 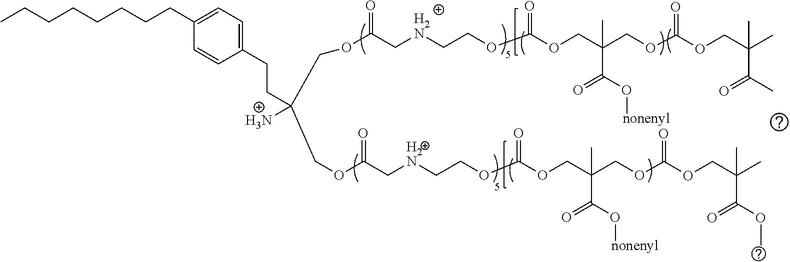 | Compound 5 |
| 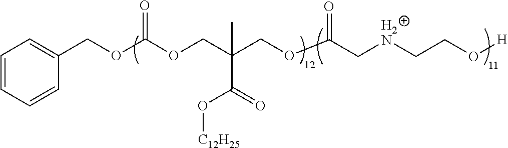 | Compound 6 |
| 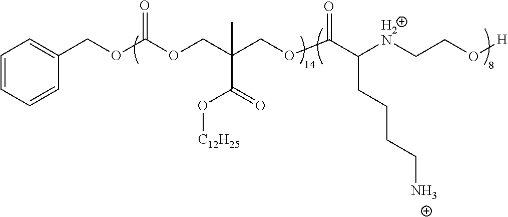 | Compound 7 |
| 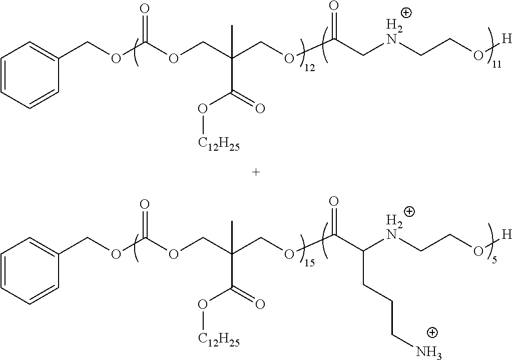 | Compound 8 |

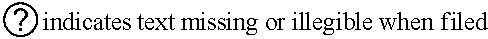

In embodiments, the compositions described here may further comprise an antibody conjugated to the co-oligomer, which may further include a linker moiety. The term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies.

In embodiments, the antibody is an anti-TROP2 antibody. Trop-2 is the protein product of the TACSTD2 gene and is a transmembrane glycoprotein upregulated in cancer cells of various types compared to non-cancer cells. Suitable anti-TROP2 antibodies include Trop-2-targeted antibody-drug conjugates and Trop-2 targeted antigen binding fragments, also referred to as Fab fragments, or “Trop-2 Fab”. In embodiments, the Trop-2-targeted antibody-drug conjugates (ADC) may comprise doxorubicin-loaded nanoparticles, a topoisomerase I inhibitor, a microtubule inhibitor such as auristatin, or an irinotecan metabolite, for example the ADC Sacituzumab govitecan (IMMU-132).

In embodiments, the compositions described here may be conjugated to an imaging agent, for example for use in methods of clinical imaging. In the context of PET imaging, radioisotope therapy, or MRI contrast imaging, the imaging agent may comprise a suitable metal ion or a metal, for example, fluorine, e.g., ¹⁸F, lutetium (Lu, e.g., ¹⁷⁵Lu or ¹⁷⁷Lu), actinium (Ac, e.g., ²¹⁷Ac, ²²⁵Ac), gallium (Ga, e.g., ⁶⁷Ga, or ⁶⁸Ga), copper (Cu), samarium (Sm), radium (Ra), yttrium (Y), palladium (Pd), iridium (Ir), gadolinium (Gd) or lead (Pb), or includes a fluorine atom-carrying moiety that may optionally function as a PET contrasting agent, by including ¹⁸F. In the context of optical imaging, the imaging agent may comprise a fluorophore such as cyanine, crystal violet, eosin, fluorescein, malachite green, Oregon green, rhodamine, and Texas Red.

In embodiments, the compositions described here may be conjugated to a radiolabeled agent, for example for use in radioimmunotherapy.

### Methods

The disclosure provides methods for delivery of a therapeutic siRNA to cancer cells, for example cancer cells expressing c-Myc. In embodiments, the cancer cells may be in vitro, ex vivo, or in vivo. The methods comprise contacting the cancer cells with a composition comprising the therapeutic siRNA non-covalently attached to a co-oligomer, as described herein. In embodiments, the methods comprise administering a pharmaceutical composition comprising a therapeutic siRNA and the copolymer to a subject in need of therapy for cancer, preferably a human subject. Accordingly, encompassed are methods of treating cancer in a subject in need thereof, the methods comprising administering to the subject a composition comprising a c-Myc directed small interference RNA (siRNA) non-covalently attached to a co-oligomer as described herein. In embodiments, the lipophilic polymer is a mixed lipophilic polymer comprising oleyl and nonenyl lipid moieties.

In embodiments, the cancer is one whose cells overexpress c-Myc compared to the expression of c-Myc in non-cancerous tissue. In embodiments c-Myc is considered to be overexpressed where its expression is elevated at least 2-fold relative to its expression in a reference non-cancerous tissue. In embodiments, the cancer is selected from the group consisting of breast cancer, liver cancer, kidney cancer, lung cancer, ovarian cancer, and bone cancer. In embodiments, the cancer is a leukemia or lymphoma. In embodiments, the cancer is refractory to standard therapy. In embodiments, the cancer is recurrent. In embodiments, the cancer is unresectable locally advanced or metastatic.

Breast cancer is the leading cause of cancer-related deaths in women. Breast cancer is divided into four subtypes: luminal A, luminal B, HER2-enriched, and triple negative breast cancer (TNBC). TNBC's 5-year survival rate is the worst of all subtypes at 76%. TNBCs may be very aggressive, metastasizing to regional sites, such as lymph nodes, or distant sites, such as other organs, decreasing 5-year survival rates to 65.4% and 12.2%, respectively. Although TNBCs may respond initially to chemo/radiotherapies, they recur at higher rates than other subtypes, and only a fraction of TNBC patients show clinical responses after immunotherapy treatment. More effective therapies against TNBC are needed. The present invention addresses this need.

Accordingly, in embodiments, the disclosure provides methods of treating breast cancer in a subject in need thereof, the methods comprising administering to the subject a composition comprising a c-Myc directed small interference RNA (siRNA) non-covalently attached to a co-oligomer as described herein. In embodiments, the lipophilic polymer is a mixed lipophilic polymer comprising oleyl and nonenyl lipid moieties. In embodiments, the breast cancer is refractory to standard therapy. In embodiments, the breast cancer is recurrent. In embodiments, the breast cancer is unresectable locally advanced or metastatic. In embodiments, the breast cancer is characterized as lacking one or more of estrogen receptor, progesterone receptor, and human epidermal growth factor receptor 2 (HER2). In embodiments, the breast cancer is characterized as lacking all three of estrogen receptor, progesterone receptor, and human epidermal growth factor receptor 2 (HER2), which cancer may also be referred to as “triple negative breast cancer” or “TNBC”.

In accordance with the methods described herein, a “subject” includes a mammal. The mammal can be e.g., any mammal, e.g., a human, primate, mouse, rat, dog, cat, cow, horse, goat, camel, sheep or a pig. Preferably, the subject is a human. The term “patient” refers to a human subject.

In embodiments of the methods described here, the subject in need of treatment may be one having a cancer that is non-responsive or refractory to, or has relapsed after, treatment with a ‘standard of care’ or first-line therapeutic agent. In this context, the terms “non-responsive” and “refractory” are used interchangeably and refer to the subject's response to therapy as not clinically adequate, for example to stabilize or reduce the size of one or more solid tumors, to slow tumor progression, to prevent, reduce or decrease the incidence of new tumor metastases, or to relieve one or more symptoms associated with the cancer. A cancer that is refractory to a particular drug therapy may also be described as a drug resistant cancer. In a standard therapy for the cancer, refractory cancer includes disease that in progressing despite active treatment while “relapsed” cancer includes cancer that progresses in the absence of any current therapy, but following successful initial therapy. Accordingly, in embodiments, the subject is one who has undergone one or more previous regimens of therapy with one or more ‘standard of care’ therapeutic agents. In such cases, the subject's cancer may be considered refractory or relapsed. In embodiments, the subject's cancer may be relapsed or recurrent.

In embodiments, the subject's cancer may be is unresectable locally advanced or metastatic.

In embodiments where the cancer is breast cancer, the breast cancer is characterized as lacking one or more of estrogen receptor, progesterone receptor, and human epidermal growth factor receptor 2 (HER2). In embodiments, the breast cancer is characterized as lacking all three of estrogen receptor, progesterone receptor, and human epidermal growth factor receptor 2 (HER2). Methods for assaying a cancer biopsy sample for the presence of one or more biomarkers, including estrogen receptor, progesterone receptor, and human epidermal growth factor receptor 2 (HER2), are known in the art. In embodiments, the methods of therapy described herein may further comprise a pre-treatment step or post-treatment step comprising assaying a tumor biopsy sample for a biomarker.

In embodiments, a composition of the present invention is further conjugated to a molecule or peptide comprising a radiometal suitable for radioisotope therapy.

“Treatment,” “treating,” and “treat” describes the management and care of a patient for the purpose of combating a disease, condition, or disorder and includes the administration of a composition described herein, either alone as monotherapy, or in combination with at least one additional API as described here, to alleviate the symptoms or complications of a disease, condition or disorder, or to eliminate the disease, condition or disorder. “Treating” or “treatment of” a condition or subject in need thereof refers to (1) taking steps to obtain beneficial or desired results, including clinical results such as an amelioration or reduction in one or more symptoms of the disease, disorder, or condition; (2) inhibiting the disease, for example, arresting or reducing the development or clinical progression of the disease, disorder, or condition, or any one or more of its clinical symptoms; (3) relieving the disease, for example, causing regression of the disease or its clinical symptoms; or (4) delaying or slowing disease progression.

In embodiments, including both monotherapy with a composition described here and combination therapies with one or more additional therapies or therapeutic agents, the administration of a composition of the invention leads to the elimination of a symptom or complication of the cancer being treated, however elimination of the cancer is not required. In one embodiment, the severity of the symptom is decreased. In the context of cancer, such symptoms may include clinical markers of severity or progression including the degree to which a tumor secretes growth factors, degrades the extracellular matrix, becomes vascularized, loses adhesion to juxtaposed tissues, or metastasizes, as well as the number of metastases and reduction in tumor size and/or volume.

According to the methods provided herein, the subject can be administered an effective amount of a composition described herein, e.g. co-oligomer complexed with siRNA. The terms “effective amount” and “effective dosage” are used interchangeably. The term “effective amount” is defined as any amount necessary to produce a desired effect, for example transfection of nucleic acid into cells and exhibiting an intended outcome of the transfected nucleic acid.

Treating cancer according to the methods described herein can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer according to the methods described herein can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer according to the methods described herein can result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×. For hematologic cancers, the count may be the number of cells related to the cancer (e.g., lymphoma or leukemia cells) in a sample of blood.

Treating cancer according to the methods described herein can result in a decrease in the number of metastatic lesions in other tissues or organs distant from the primary tumor site. Preferably, after treatment, the number of metastatic lesions is reduced by 5% or greater relative to the number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer according to the methods described herein can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time. In one embodiment, after treatment the tumor growth rate may be about zero and is determined to maintain the same size, e.g., the tumor has stopped growing.

Treating cancer according to the methods described herein can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped

In the context of the present methods, the term “administering” when used in connection with a composition described herein may refer to direct administration or indirect administration, which encompasses the act of prescribing a composition of the disclosure. Direct administration includes administration to cells in vitro, administration to cells in vivo, administration to a patient by a medical professional or self-administration by the patient. When used herein in reference to a cell, refers to contacting a composition with the cell, for example, by addition of the composition to the cell culture media or via injection.

The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Administering can also be performed, for example, once, a plurality of times, and/or over one or more extended periods.

In embodiments, a composition of the invention is administered by an intravenous route.

The dosage and frequency (single or multiple doses) administered to a subject can vary depending upon a variety of factors, for example, whether the subject suffers from another disease, its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compositions described herein including embodiments thereof. Adjustment and manipulation of established dosages (e.g., frequency and duration) are well within the ability of those skilled in the art.

In embodiments, a composition can be administered systemically or locally (e.g. intratumoral injection, intravenous injection) at intervals of 6 hours, 12 hours, daily or every other day or on a weekly or monthly basis to elicit the desired benefit or otherwise provide a therapeutic effect.

In embodiments, the compositions described here may be used in combination therapy with one or more additional anti-cancer therapies or therapeutic agents. The term “combination therapy” or “co-therapy” includes the administration of a therapeutically effective amount of a composition of the invention as part of a treatment regimen intended to provide the beneficial effect from the co-action of the composition and at least one additional anti-cancer therapy or therapeutic agent, which may be referred to as an “active pharmaceutical ingredient” (“API”). “Combination therapy” is not intended to encompass the administration of two or more therapeutic compounds as part of separate monotherapy regimens that incidentally and arbitrarily result in a beneficial effect that was not intended or predicted.

In embodiments, the methods of treating breast cancer described here may further comprise administering an immunotherapy agent to the subject. In embodiments, the immunotherapy agent is an immune checkpoint inhibitor such as a programmed cell death protein 1 (PD-1) or programmed cell death ligand-1 (PD-L1) inhibitor. In embodiments, the PD-1 or PD-L1 inhibitor is selected from the group consisting of pembrolizumab, nivolumab, cemiplimab, dostarlimab, atezolizumab, avelumab, and durvalumab.

In embodiments, the methods of treating breast cancer described here may further comprise administering a PARP inhibitor, for example olaparib, rucaparib, or talazoparib.

Preferably, the administration of a composition of the invention in combination with one or more additional APIs, such as an immunotherapy agent, as discussed herein provides a synergistic response in the subject being treated. In this context, the term “synergistic” refers to the efficacy of the combination being more effective than the additive effects of either single therapy alone. The synergistic effect of a combination therapy according to the disclosure can permit the use of lower dosages and/or less frequent administration of at least one agent in the combination compared to its dose and/or frequency outside of the combination. Additional beneficial effects of the combination can be manifested in the avoidance or reduction of adverse or unwanted side effects associated with the use of either therapy in the combination alone (also referred to as monotherapy).

In the context of combination therapy, administration a composition of the invention may be simultaneous with or sequential to the administration of the one or more additional active agents, such as an immunotherapy agent. In another embodiment, administration of the different components of a combination therapy may be at different frequencies.

The additional API(s) can be formulated for co-administration with a composition of the invention in a single dosage form. The additional API(s) can also be administered separately from the dosage form that comprises a composition of the invention. When the additional active agent is administered separately, it can be by the same or a different route of administration, and/or at the same or different time.

In embodiments, the at least one additional API may be a BCL-2 pathway inhibitor, a protein kinase inhibitor, a PD-1/PD-L1 inhibitor, a checkpoint inhibitor, a platinum based anti-neoplastic agent, a topoisomerase inhibitor, a nucleoside metabolic inhibitor, an alkylating agent, an intercalating agent, a tubulin binding agent, an inhibitor of DNA repair, and combinations thereof. In embodiments, the at least one additional API is a BCL-2 pathway inhibitor or a PD-1/PD-L1 inhibitor.

In embodiments, a composition of the present invention is further conjugated to an imaging agent and the methods comprise clinical imaging of a subject in need thereof. In embodiments, the imaging may comprise a technique selected from positron emission tomography (PET), single photon emission computer tomography (SPECT), magnetic resonance imaging (MRI), contrast aided (e.g., gadolinium contrast) magnetic resonance imaging (cMRI), and fluorescence (FL) or absorbance-based optical imaging.

In the context of PET imaging, radioisotope therapy, or MRI contrast imaging, the imaging agent may comprise a suitable metal ion or a metal, for example, fluorine, e.g., ¹⁸F, lutetium (Lu, e.g., ¹⁷⁵Lu or ¹⁷⁷Lu), actinium (Ac, e.g., ²¹⁷Ac, ²²⁵Ac), gallium (Ga, e.g., ⁶⁷Ga, or ⁶⁸Ga), copper (Cu), samarium (Sm), radium (Ra), yttrium (Y), palladium (Pd), iridium (Ir), gadolinium (Gd) or lead (Pb), or includes a fluorine atom-carrying moiety that may optionally function as a PET contrasting agent, by including ¹⁸F.

In the context of optical imaging, the imaging agent may comprise a fluorophore such as cyanine, crystal violet, eosin, fluorescein, malachite green Oregon green, rhodamine, and Texas Red.

### Pharmaceutical Compositions

The disclosure also provides pharmaceutical compositions comprising a composition as described herein. The pharmaceutical compositions may contain pharmaceutically acceptable excipients or additives which may vary depending on the intended route of administration. Examples of such excipients or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additional acceptable carriers, excipients, or stabilizers may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Formulation of the pharmaceutical compositions of the present disclosure can vary according to the route of administration selected (e.g., solution, emulsion).

In some embodiments, the composition can include a cryoprotectant agent. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

While various embodiments and aspects of the present disclosure are shown and described herein, it will be obvious to those skilled in the art that such embodiments and aspects are provided by way of example only. Numerous variations, changes, and substitutions will now occur to those skilled in the art without departing from the disclosure. It should be understood that various alternatives to the embodiments of the disclosure described herein may be employed in practicing the disclosure.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

Although genetic inactivation of c-MYC has been shown to produce rapid tumor regression and re-establishment of anti-cancer immunity in several model systems, pharmacological-mediated MYC inhibition has proven difficult. MYC has been described as “undruggable” because of its nuclear localization and lack of defined enzymatic activity. Attempts have been made to target transcriptional MYC regulators, its heterodimerization with MAX, its DNA-binding capability, or its phosphorylation to promote degradation, all with limited success. A recent anti-sense approach, DCR-MYC, which was a first-in-class Dicer-substrate small interfering RNA targeting MYC, was ineffective due at least in part to lack of delivery specificity to tumors resulting in toxicity.

Described here are improved compositions and methods for targeted delivery of anti-MYC siRNA to tumors in the treatment of cancer, using breast cancer as a model system.

A new MYC-targeting therapeutic agent was produced to target the MYC oncogene in triple negative breast cancer (TNBC). The agent consisted of a MYC-targeting small interfering RNAs (siRNAs) and a co-oligomer of cationic alpha aminoester and lipophilic monomer repeating units, referred to herein as a Charge-Altering Releasable Transporter (CART). CARTs are amphiphilic block co-oligomers with high gene delivery efficacy in vitro and in vivo providing higher percent transfection than commercially available agents. CARTs can change their electrostatic properties from polycationic to neutral, as described in Blake et al. Chem. Sci (2020) 11:2951. In addition, CARTs show no significant cytotoxicity compared to other cationic polymeric gene delivery vectors. Depending on the CART structure, CART-RNA nanoparticles are able to enter different types of cells and tissues with remarkable selectivity. Described below are CARTs with high tropism for breast cancer cells that effectively deliver anti-MYC siRNAs (siMYC) intracellularly. In addition, CART siMYC intratumoral injection results in reduced MYC expression and reduced MYC-dependent signaling which in turn impacts cell cycle progression and protein translation of cells of primary TNBC tumors. Furthermore, CART siMYC also reduced growth of untreated distal tumors suggesting that CART siMYC may be an effective anti-MYC therapeutic with abscopal effects.

### Identification of CARTs Specific for Breast Cancer Cells

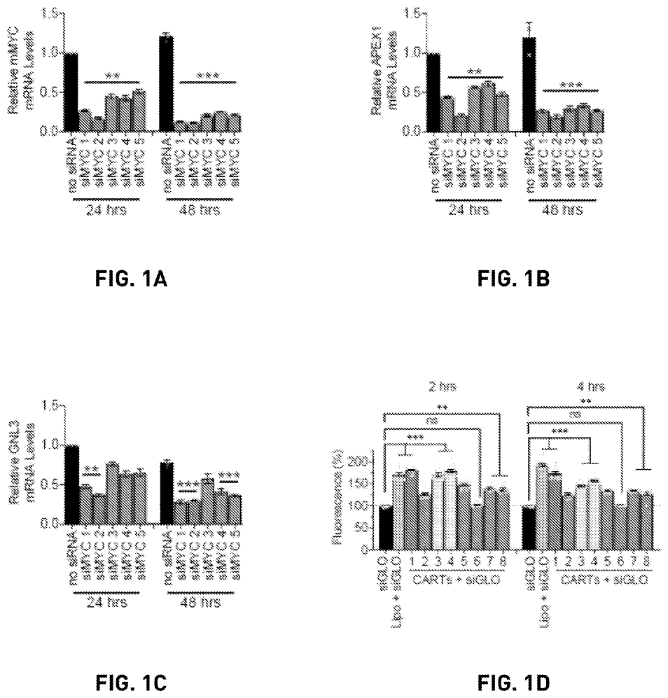

In preliminary experiments, the best acting CART and the most effective MYC-targeting siRNA were identified by testing panels of CARTs and siRNA molecules. A panel of 5 different siRNAs targeting exon 1 of mouse c-Myc mRNA was tested. Out of the five siRNAs, the first two murine siRNAs (referred to herein as “siMYC 1” SEQ ID NO: 2 and “siMYC 2” SEQ ID NO: 3) were the most effective, decreasing c-Myc mRNA levels by at least 70% (FIG. 1A). Importantly, siMYC 1 and 2 also decrease Myc transcriptional function as assessed via the expression of two Myc-target genes Apex1 and Gnl3 (FIG. 1B, FIG. 1C). Thus, siMYC 1 and 2 were used in subsequent experiments.

To identify the best acting CART against the TNBC cell 4T1, a panel of 8 different CARTs was evaluated and compared side by side to lipofectamine, a well-known lipid-based transfection reagent. CARTs 1, 3, and 4 were able to deliver the fluorescent cargo siGLO at 2 and 4 hours post treatment with similar efficiency as lipofectamine (FIG. 1D). CART 1 performed the best at both time points.

CARTs 2, 5, 7, 8 were less efficient delivery vehicles compared to CARTs 1, 3, and 4. CART 6 was unable to deliver its siRNA cargo to these cells.

TABLE 3
Physical Characteristics of an Exemplary Compositions:
Z-average size, the distribution size, the Polydispersity
index (PDI), and the Zeta potential for CART 1 loaded
with five different siMYC molecules or CART 6 (which
does not bind) TNBC cells, loaded with siMYC.

| Composition | Z (nm) | Distribution (nm) | PDI | Zeta (mV) |
|---|---|---|---|---|
| CART 1 + | 169 | 162 | 0.20 | 31 |
| siMYC 1 | | | | |
| CART 1 + | 186 | 171 | 0.23 | 33 |
| siMYC 2 | | | | |
| CART 1 + | 270 | 264 | 0.20 | 19 |
| siMYC 3 | | | | |
| CART 1 + | 177 | 180 | 0.17 | 26 |
| siMYC 4 | | | | |
| CART 1 + | 211 | 198 | 0.30 | 27 |
| siMYC 5 | | | | |
| CART 6 + | 189 | 177 | 0.23 | 26 |
| siMYC 2 | | | | |

### CART siMYC Reduces the Viability of Human and Mouse TNBC Cells

Next, the Myc-targeting siRNAs 1 and 2 were combined with the best acting CARTs, CART 1 and CART 3, and assessed for their ability to reduce the viability of Myc-high expressing 4T1 TNBC cells. 48 hours post treatment, there was an initial decrease on TNBC cell viability by CART 1 siMYC 2 and CART 3 siMYC 2 relative to either CART alone or CART 1 or 3 with a scramble siRNA control (siCTL). The maximal inhibitory effect of CART 1 or 3 with siMYC 2 was achieved at 96 hours post treatment where viability was reduced by 50%. Lipofectamine loaded with siMYC 2 reduced TNBC cell viability 80%, and a similar reduction was seen with the bromodomain inhibitor JQ1—a well-known anti-cancer drug.

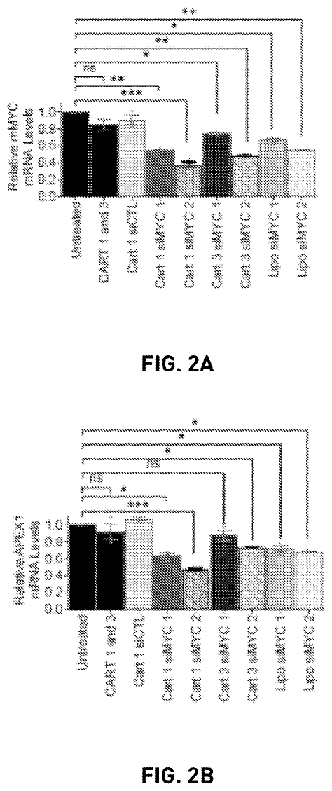

Myc mRNA expression in TNBC cells treated with CART 1 siMYC 2 were reduced by 60% compared to a reduction of 50% by CART 3 siMYC 2 and 45% by lipofectamine loaded with siMYC 2 (FIG. 2A). The impact of CART/siMYC complexes on Myc signaling was assessed using the mRNA levels of the Myc target Apex1. As expected CARTI siMYC 2 efficiently decreased Apex1 expression by 50% while other treatments decreased Apex1 expression by 20% or had no effect on Apex1 levels (FIG. 2B).

These results show that CART 1 siMYC-2 is more effective than lipofectamine loaded with siMYC 2 at decreasing Myc RNA levels and reducing Myc signaling in TNBC cells while at the same time it is less effective at reducing cell viability. This may be due to lipofectamine having Myc-independent effects in TNBC cells that account for the reduction in cell viability.

Subsequent experiments were performed with CART 1 siMYC 2, which is referred to in the following as simply “CART siMYC”.

Global transcriptional changes induced by CART siMYC in TNBC cells was compared to cells treated with CART carrying a scramble control siRNA (siCTL) or cells treated with DMSO. Compared to CART siCTL, CART siMYC treatment resulted in a significant decrease in Myc signaling which resulted in a marked reduction in cell cycle progression and protein translation as assessed via GSEA analyses. In addition, CART siMYC differentially affected pathways related to ribosomal synthesis, mRNA binding, and protein translation relative to CART siCTL as determined by GO term enrichment analyses (data not shown). CART siCTL did not induce significant changes in the transcriptome of TNBC cells relative to vehicle, indicating that the CART vehicle itself does not induce changes in ribosomal biology and protein translation.

### The Therapeutic Efficacy of CART siMYC Relies on TNBC Cells Expressing High MYC Levels

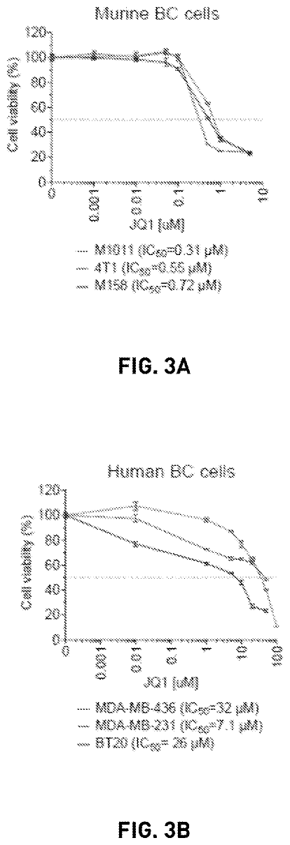

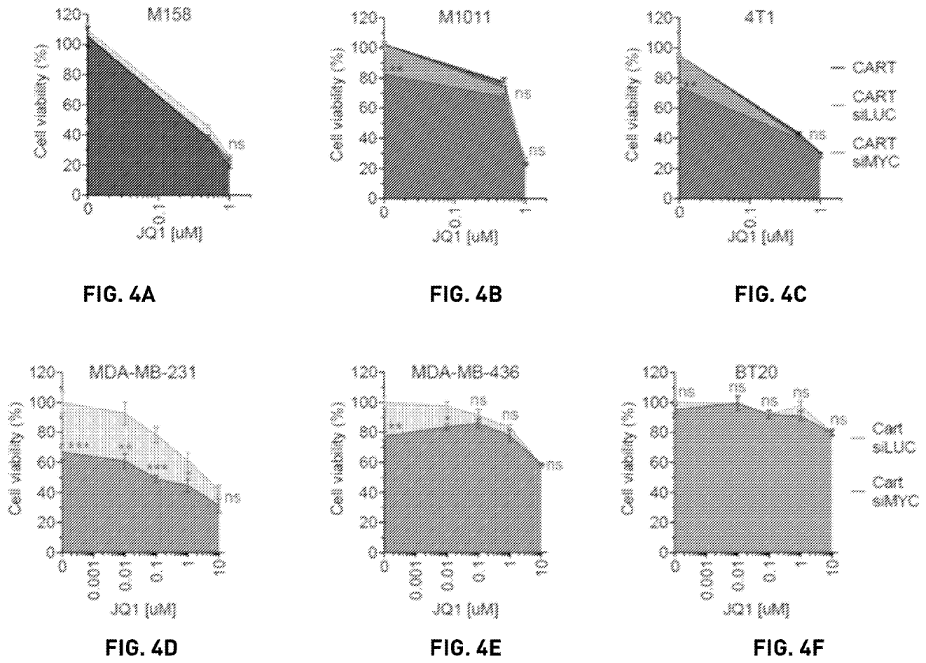

The specificity and dependence of CART siMYC's inhibitory effect on Myc or MYC levels in murine or human TNBC cells, respectively, was evaluated next. To titrate and lower Myc levels in TNBC cells we used the BETi JQ1 which is known to decrease Myc expression in cancer cells by inhibiting the transcription factor BRD438. A number of different murine and human TNBC cell lines expressing high (murine cells: M158, M1011, and 4T1; and human cells: MDA-MB-231 and MDA-MB-436) or low MYC levels (human cell: BT20) were evaluated. First, the IC50 for the various cell lines following a 24 hour treatment with JQ1 was determined. Murine TNBC cells showed an IC50 of 0.3-0.72 μM while human TNBC cells showed an IC50 between 7-32 μM (FIG. 3A and FIG. 3B). Next, we pre-treated TNBC cells with JQ1 at concentrations ranging from 0-10 μM for 2 hours followed by a 48 hour treatment with CART alone, CART siLUC (our control siRNA), or CART siMYC added to JQ1-containing media. M158 cells, which express the highest levels of Myc among the murine TNBC cells, showed the highest sensitivity to CART siMYC with viability dropping to 45% relative to CART siLuc (FIG. 4A). The viability of M1011 and 4T1 cells was reduced by 25% upon treatment with CART siMYC but not with CARTs alone or with CART siLUC (FIG. 4B, FIG. 4C). In human TNBC cells, treatment with a human MYC-targeting siRNA (SEQ ID NO: 1) reduced viability by 35% or 22% in MDA-MB-231 or MDA-MB-436 cells, respectively (FIG. 4D, FIG. 4E). No reduction in cell viability was observed in the low MYC-expressing BT20 cells (FIG. 4F). These results show that pre-treatment of murine and human TNBC cells with JQ1, to decrease Myc/MYC mRNA levels, decreases the inhibitory effect of CART siMYC in a concentration dependent manner in all TNBC cells except for BT20, which expresses low MYC levels and are not significantly affected by JQ1 at the concentrations used. We conclude that CART siMYC is effective against TNBC cells expressing high, but not low, MYC levels and that the breadth of its therapeutic effects is dependent on cellular MYC levels. This indicates that therapy with CART siMYC will exhibited targeted effects against MYC-overexpressing tumor cells, as opposed to normal cells.

### Intratumoral Injection of CART siMYC is the Most Effective Delivery Route In Vivo

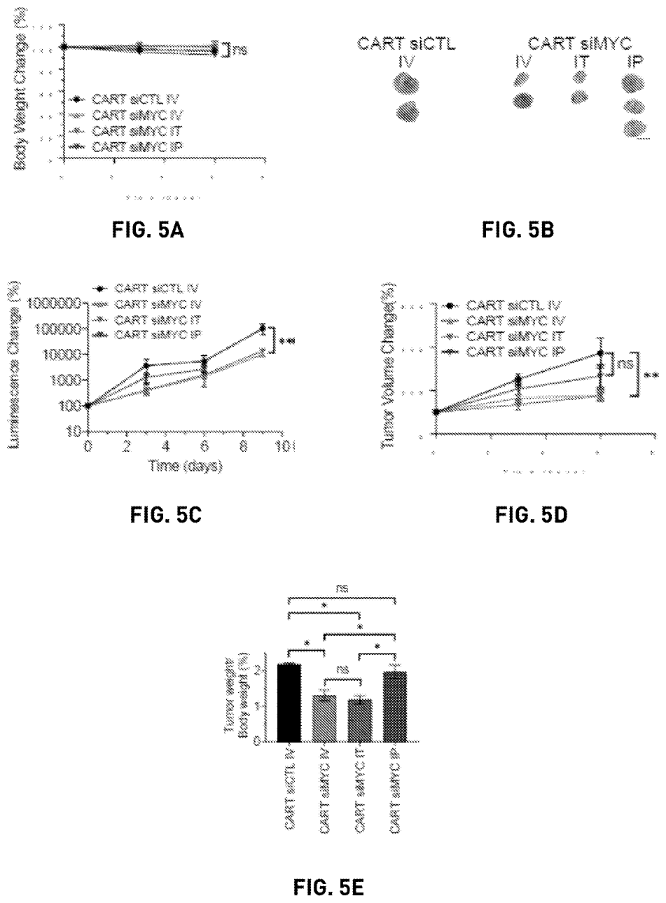

Next, the therapeutic efficacy of CART siMYC over CART siCTL at decreasing the growth of TNBC tumors was evaluated. The best delivery method for the CART siMYC conjugates was first determined. Delivery of CART siMYC intratumorally, intravenously, or intraperitoneally did not induce changes in body weight (FIG. 5A). Intratumoral and intravenous injections of CART siMYC effectively decreased the growth of TNBC tumors relative to CART siCTL delivered intravenously as assessed by gross tumor images (FIG. 5B), tumor growth over time based on luminescence (FIG. 5C) and tumor volume readings (FIG. 5D), and tumor weight at the time of euthanasia (FIG. 5E). We conclude that CART siMYC can be effectively delivered via intravenous and intratumoral routes.

### CART siMYC Reduced Local and Distal TNBC Tumor Growth

CART siMYC was effective at decreasing TNBC cell viability in vitro and when delivered intratumorally or intravenously, it decreased tumor growth. Next, the ability of CART siMYC to reduce tumor growth in locally treated tumors compared to adjacent untreated tumors was evaluated. Since phenotypic and molecular changes locally upon CART siMYC treatment were being assessed, the complex was delivered intratumorally.

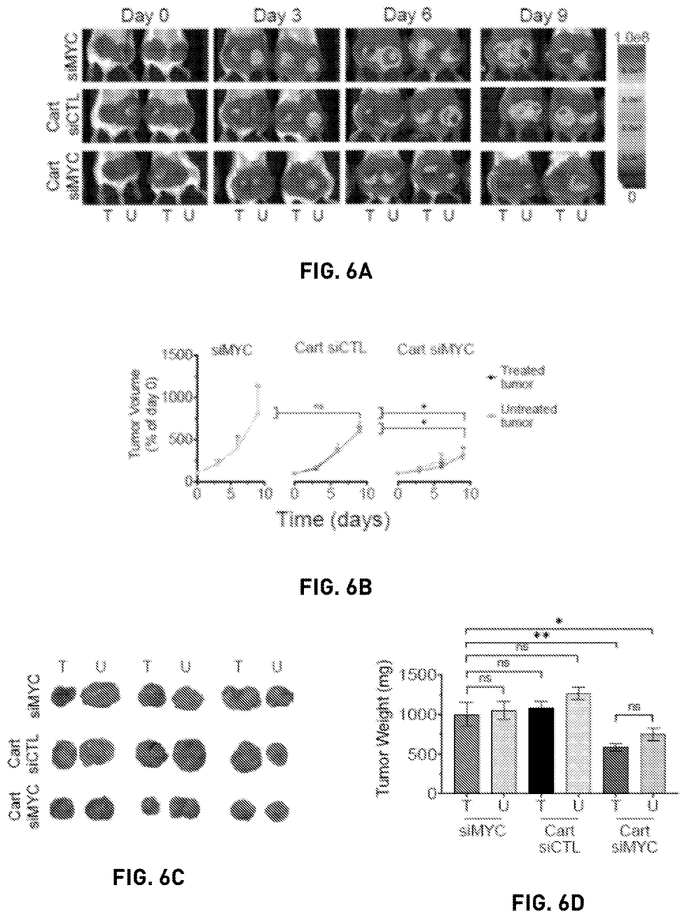

9-day short-term treatment with CART siMYC reduced the growth of locally treated TNBC tumors compared to CART siCTL or siMYC alone as assessed by BLI imaging (FIG. 6A), tumor volume over time (FIG. 6B, gross tumor images (FIG. 6C), and tumor weight at the time of euthanasia (FIG. 6D). The growth of the untreated TNBC tumor was also reduced in CART siMYC treated mice compared to CART siCTL or siMYC alone suggesting an abscopal effect.

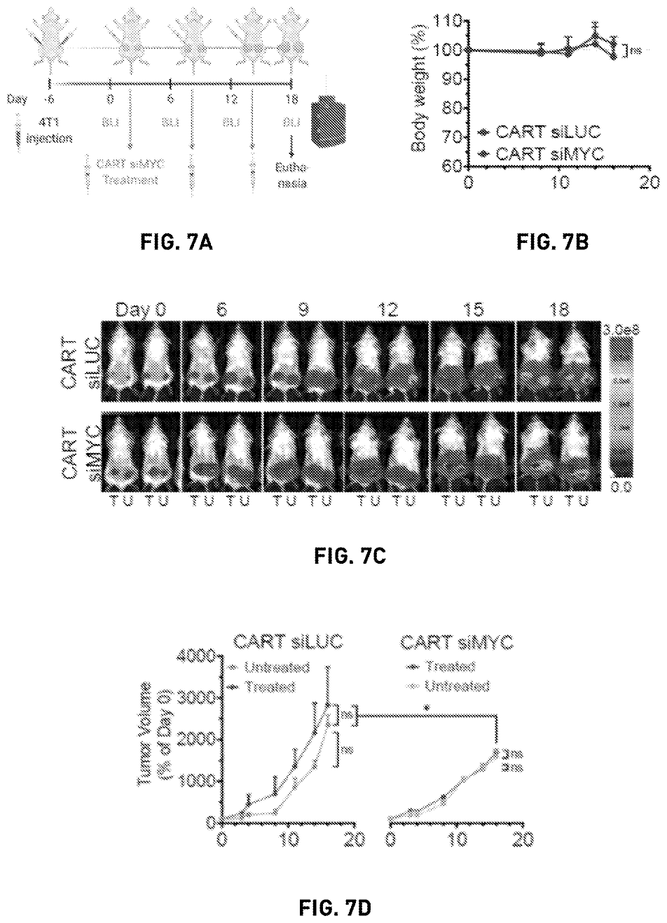

A longer-term 18-day treatment was also performed in mice bearing two TNBC tumors, the subcutaneous tumor in the left was treated with CART siRNA while the tumor on the right remained untreated, the aim being to test the ability of CART siMYC to induce an abscopal effect (FIG. 7A). 18-day treatment with CART siMYC did not affect body weight over time (FIG. 7B) but significantly reduced the growth of both the locally treated (T) tumor and the adjacent untreated (U) tumor compared to CART siLUC which did not affect tumor growth (FIG. 7C, FIG. 7D).

### CART siMYC Decreased Myc Expression and Signaling in Locally Treated TNBC Tumors

Transcriptome analyses of locally treated tumors show the ability of CART siMYC over CART siLUC to change the transcriptome of TNBC cells and to reduce Myc expression and Myc signaling (data not shown). CART siMYC but not CART siLuc also reduced cell cycle progression and protein translation in vivo in TNBC tumors indicating that intratumoral injection of CART siMYC effectively targets the Myc pathway in vivo.

### Intratumoral Injection of CART siMYC Also Reduced Myc Levels and Signaling in Local Treated Tumors Compared to CART siLUC

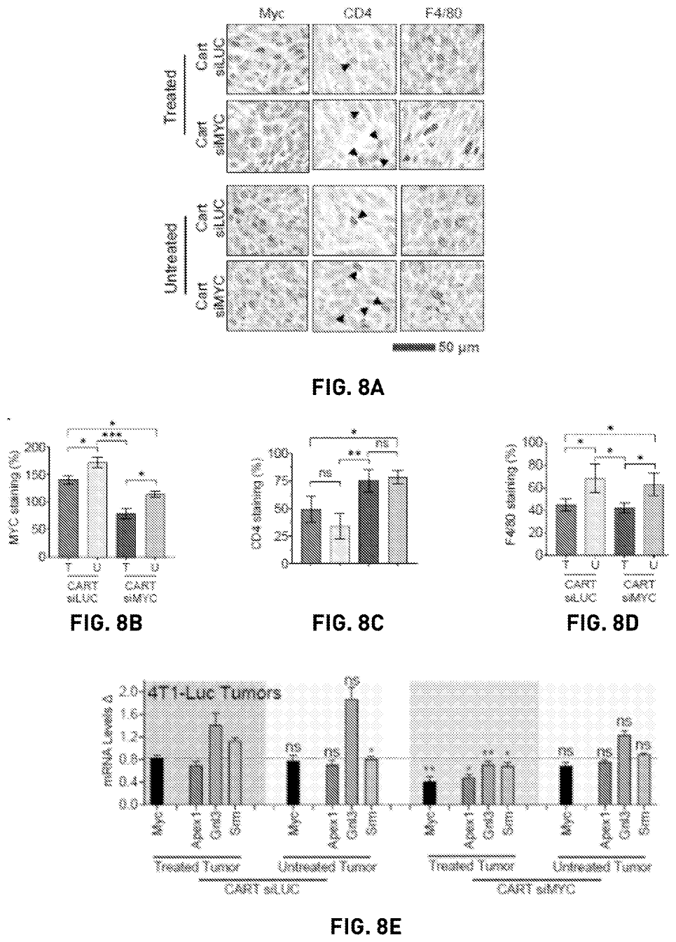

Further analyses using RTqPCR and immunohistochemistry show that Myc mRNA and protein levels are decreased in local treated tumors compared to distal untreated tumors of CART siMYC-treated mice (FIG. 8A, FIG. 8B and FIG. 8E), but Myc expression in both tumors of CART siMYC-treated mice was lower compared to the respective tumors in CART siLUC-treated mice. Of note, the abundance of CD4+ immune cells increased in both local and distal TNBC tumors of CART siMYC-treated mice relative to CART siLUC-treated mice (FIG. 8C) while the abundance of F4/80+ immune cells is similar between both treatment groups (FIG. 8D). Importantly, neither CART siCTL nor siMYC alone decreased Myc protein levels in either local or distal TNBC tumors and there was no induced recruitment of CD4+ or F4/80+ immune cells in these controls. We conclude that CART siMYC induces the tumor recruitment of CD4+ immune cells in local treated and distal untreated TNBC tumors without affecting Myc expression in distal tumors.

The MYC oncogene is known as a universal driver of cancer cell proliferation and immune evasion. MYC is overexpressed in 53% of TNBCs. Here, we describe the development of a new MYC-targeting therapeutic using CART technology and siRNAs that is highly effective at inhibiting MYC signaling in TNBC tumors and reducing their growth. Unexpectedly, this therapy was also effective to inhibit the growth of untreated tumors in the same animal, without decreasing MYC levels in those tumors. Without being bound by any theory, by decreasing MYC expression, CART siMYC may allow tumor recruitment of immune cells to the local treated tumor and also activate a more global anti-tumor immune response that allows immune cell recruitment to distal untreated tumors.

These pre-clinical studies have clinical implications for the treatment of local and metastatic TNBC since CART siMYC can be delivered directly to primary TNBC tumors reducing MYC levels and resulting in reduced tumor growth and also overcoming MYC-induced immune evasion which serves to activate anti-tumor immune responses globally. In addition, since CART siMYC appears to have an abscopal effect, it may synergize with immune checkpoint inhibitors that can further enhance anti-tumor immunity. Third, CARTs have the potential to be altered to become “bispecific” drugs that could prevent receptor signaling and also deliver siRNAs to target the MYC oncogene. For example, CART siMYC can be coated with antibodies that will bind to the TNBC specific receptor TROP2 (e.g. Sacituzumab govitecan) preventing TROP2-mediated cell cycle progression and also reducing MYC signaling. An alternative use of CARTs is as a theranostic tool. CARTs may be loaded with imaging probes and siRNAs allowing simultaneous imaging and treatment of TNBC tumors. In summary, CART siMYC is a novel drug to target the MYC oncogene in TNBC with the potential to induce an immune-mediated abscopal effect.

### Methods

Animals: Animals were identically raised and housed at Stanford University following Administrative Panel on Laboratory Animal Care (APLAC) guidelines and procedures. 5-6 week old BALB/c female mice were used to grow subcutaneous and orthotopic 4T1 tumors. FVB/N-Tg(MMTV-PyVT)634 Mul/J mice develop mammary tumors at around day 55 at which point treatment was started. Mice genotypes were confirmed by PCR of genomic DNA from animal tails.

Cell culture: Murine 4T1, M158, and M1011 cell lines and human MDA-MB-231, MDA-MB-468, and BT20 cell lines were grown in RPMI supplemented with 10% FBS, 1% penicillin/streptomycin, 1% Glutamax, 1% non-essential amino acids, and 1% pyruvate at 37° C. in 5% CO2. To reduce the levels of MYC in these cells lines in a concentration dependent manner, we used the Bromodomain inhibitor JQ1 which is known to indirectly target the MYC oncogene34.M158 and M1011 cells were a gift from Dr. Lewis A. Chodosh, the rest of the cell lines were acquired from ATCC.

Bioluminescence Imaging: Mice were imaged as previously described. Briefly, female BALB/c mice bearing 4T1-LUC tumors were imaged at days 0, 3, 6, 9, 12, 15, and 18 days after treatment with CART siLUC, CART siCTL, or CART siMYC. Prior to bioluminescence imaging, mice were anesthetized using 2% isoflurane and injected intraperitoneally with D-luciferin at 150 mg/kg. 5 minutes after D-luciferin injection, mice were visualized using an in vivo bioluminescence/optical imaging system (Ami HT, Spectral Instruments Imaging). Image analysis was performed using Ami View software (V1.7.06, Spectral Instruments Imaging).

Chemical characterization of CARTs: Nanoparticle sizes were measured using dynamic light scattering (DLS). Polydispersity index and cZeta potential measurements were conducted using electrophoretic light scattering (ELS). Each value is the average of 2 measurements.

CellTiter Glo assay: A total of 2,000 4T1, M158, M1011, MDA-MB-231, MDA-MB-468, or BT20 cells were plated in a 96-well clear-bottom plates, allowed to attach overnight and treated with different concentrations of JQ1 to identify the IC50 for each specific cell line. For studies using CART siCTL or CART siMYC along with JQ1, cells were seeded at described above and they were treated with JQ1 at 0, 0.5, and 1 uM for 2 hours before CART siCTL or CART siMYC were added to cells at a concentration of 0.2 mM for CARTs and 0.1 μg/μl for siCTL or siMYC. Plates were incubated for 48 hours and upon treatment completion, a CellTiTer Glow assay was performed according to manufacturer's instructions (Promega). 96-well plates were rocked for 5 minutes at room temperature, 100 μl from each well were transferred to a white opaque 96-well plate and luminescence was read using a SpectraMax Paradigm Multi-Mode Detection Platform plate reader (Molecular Devices).

Real-time quantitative PCR: We seeded 6-well plates with 4T1 cells at 80% confluency and treated with a vehicle control, siCTL or siMYC alone, a mix of CARTI and 3, and combinations of Lipofectamine, CART 1 or CART3 with siMYC 1 or 2 or siCTL. for 48 hours at 5% CO2 in a 37° C. incubator. After treatment, 4T1 cells were collected and pelleted, and RNA was isolated using the RNeasy Plus Mini Kit (Qiagen) according to manufacturer's instructions. RNA isolation from breast cancer tumors were isolated with the same kit except that we adjusted the protocol for tissue processing. Following RNA isolation, equal amounts of RNA were used to synthesize cDNA using SuperScriptIII (ThermoFisher) and real-time quantitative PCR was performed using SYBR Green pPCR kit (Roche) using a HT7900 Real-Time PCR system with QuantStudio12K Flex software (Applied Biosystems). Each sample was run in triplicates and raw data was processed using the cycle threshold method were samples were normalized using

Data Analysis: Downstream analysis was performed using a combination of programs including STAR, HTseq, Cufflink and our wrapped scripts. Alignments were parsed using Tophat program and differential expressions were determined through DESeq2/edgeR. GO and KEGG enrichment were implemented by the ClusterProfiler. Gene fusion and difference of alternative splicing event were detected by Star-fusion and rMATS software.

Reads mapping to the reference genome: Reference genome and gene model annotation files were downloaded from genome website browser (NCBI/UCSC/Ensembl) directly. Indexes of the reference genome was built using STAR and paired-end clean reads were aligned to the reference genome using STAR (v2.5). STAR used the method of Maximal Mappable Prefix (MMP) which can generate a precise mapping result for junction reads.

Quantification of gene expression level: HTSeq v0.6.1 was used to count the read numbers mapped of each gene. And then FPKM of each gene was calculated based on the length of the gene and reads count mapped to this gene. FPKM, Reads Per Kilobase of exon model per Million mapped reads, considers the effect of sequencing depth and gene length for the reads count at the same time, and is currently the most commonly used method for estimating gene expression levels36.

Differential expression analysis: (For DESeq2 with biological replicates) Differential expression analysis between two conditions/groups (two biological replicates per condition) was performed using the DESeq2 R package (2_1.6.3). DESeq2 provide statistical routines for determining differential expression in digital gene expression data using a model based on the negative binomial distribution. The resulting P-values were adjusted using the Benjamini and Hochberg's approach for controlling the False Discovery Rate (FDR). Genes with an adjusted P-value <0.05 found by DESeq2 were assigned as differentially expressed. (For edgeR without biological replicates) Prior to differential gene expression analysis, for each sequenced library, the read counts were adjusted by edgeR program package through one scaling normalized factor. Differential expression analysis of two conditions was performed using the edgeR R package (3.16.5). The P values were adjusted using the Benjamini & Hochberg method. Corrected P-value of 0.05 and absolute foldchange of 1 were set as the threshold for significantly differential expression. The Venn diagrams were prepared using the function vennDiagram in R based on the gene list for different group.

Correlations: To allow for log adjustment, genes with 0 FPKM are assigned a value of 0.001. Correlation were determined using the cor.test function in R with options set alternative=“greater” and method=“Spearman”.

Clustering: To identify the correlation between difference, we clustered different samples using expression level FPKM to see the correlation using hierarchical clustering distance method with the function of heatmap, SOM (Self-organization mapping) and kmeans using silhouette coefficient to adapt the optimal classification with default parameter in R.

GO and KEGG enrichment analysis of differentially expressed genes: Gene Ontology (GO) enrichment analysis of differentially expressed genes was implemented by the clusterProfiler R package, in which gene length bias was corrected. GO terms with corrected P-value less than 0.05 were considered significantly enriched by differential expressed genes. KEGG is a database resource for understanding high-level functions and utilities of the biological system, such as the cell, the organism and the ecosystem, from molecular level information, especially large-scale molecular datasets generated by genome sequencing and other high-through put experimental technologies (http://www.genome.jp/kegg/). We used clusterProfiler R package to test the statistical enrichment of differential expression genes in KEGG pathways.

PPI analysis of differentially expressed genes: PPI analysis of differentially expressed genes was based on the STRING database, which contained known and predicted Protein-Protein Interactions. For the species existing in the database (like human and mouse), we constructed the networks by extracting the target gene lists from the database.

Differentially expressed gene annotation: TFCat and Cosmic database were used to annotate the differential expressed gene. TFCat is a curated catalog of mouse and human transcription factors (TF) based on a reliable core collection of annotations obtained by expert review of the scientific literature. COSMIC is a database designed to store and display somatic mutation information and related details which contains information relating to human cancers.

In vivo experiments: 5-6 week old BLAB/c female mice were used in all in vivo studies. Balb/c mice were subcutaneously injected with 10,000 4T1 cells expressing firefly luciferase (LUC) in the lower left flank and the lower right flank. After tumors reached a tumor volume of 200-300 mm3, mice were assigned to different treatment groups which include: siMYC alone, CART alone, CART siLUC, CART siCTL, or CART siMYC. Mice were treated every 3 days with each treatment which were delivered intratumorally. CARTs were used at 5 μg and siRNAs were used at 1 μg/μl. To make the formulation, siRNAs were added to 1×PBS pH 5.5 and CARTs dissolved in DMSO were added right before injection in mice. After adding CARTs to siRNA-PBS solution, tubes were flicked vigorously until the solution turns hazy suggesting formation of CART vesicles. Nine days or eighteen days after treatment, mice were given a second dose of their respective treatments and they were euthanized 24 hours after to weight tumors and collect plasma, and other tissues.

Immunohistochemistry (IHC): 4T1-Luc breast cancer tumors were treated with siMYC alone, CART siLUC, CART siCTL, or CART siMYC for 9 days were retrieved from mice, immersed and fixed in 10% formalin for 24 h, and transferred to 70% ethanol. Tissues were embedded in paraffin using standard procedures on a Tissue-TEK VIP processor (Miles Scientific). From these paraffin blocks, 5 μm sections were mounted on Apex superior adhesive slides (Leica Microsystems) and stained as previously described37. Stained IHC sections were mounted with antifade mounting medium (Pro-Long Gold; Invitrogen) and coverslips were used to seal the slides, Images from slides were acquired at 25° C. on a Zeiss Axiovert 200 M inverted confocal microscope with a 40 Plan Neofluor objective using IP Lab 4.0 software (Scanalytics). Antibodies used for IHCs include: human MYC (Millipore Sigma c-Myc (EP121)), CD4 (abcam, ab183685, EPR19514), F4/80 (ThermoScientific, MF48000), and F-LUC (ThermoScientific, MA1-16880). The same protocol was used to process untreated breast ncacer tumors, spleens, and lymph nodes from mice treated with different CART siRNA combinations.

Toxicity studies: 250-300 μl of blood were collected via the retro-orbital route from female BALB/c mice bearing 4T1-LUC tumors after 18 day treatment with siMYC alone, CART siLUC, CART siCTL, or CART siMYC. Mice were given 500 μl of saline intraperitoneally to help with blood recovery. Fresh blood was collected from the same mice and submitted to the Animal Diagnostic Laboratory at Stanford University to measure plasma levels of different circulating factors including: alanine aminotransferase (ALT), aspartate transaminase (AST), alkaline phosphatase (Alk. Phos.), Billirubin, and gamma-glutamyl transferase (GGT), cholesterol, triglycerides, sodium, potassium, high-density lipoprotein (HDL), low-density lipoprotein (LDL), chloride, CO2, glucose, albumin, creatinine, total protein, blood urea nitrogen (BUN), calcium, and globulin.

Statistical analyses: Graphpad prism (GraphPad Prism software) was used to generate graphs and to perform statistical analyses. These analyses were performed using an unpaired t-test assuming Gaussian distribution with Welch's correction without assuming equal standard deviations for all comparisons between two different treatment groups. Error bars represent standard deviations (SD) or standard error of the mean (SEM) as specified in each figure legend. A probability value (P) of 0.05 or lower was considered significant and is indicated by one asterisk (*) while P<0.01 is indicated by two asterisks (**), P<0.005 is indicated by three asterisks (***), and P<0.001 is indicated by four asterisks (****).

1. A composition comprising a c-Myc directed small interference RNA (siRNA) non-covalently attached to a co-oligomer of cationic alpha aminoester and lipophilic monomer repeating units, wherein the lipophilic monomer comprises oleyl and nonenyl lipid moieties, wherein the co-oligomer is selected from the group consisting of Compound 1, Compound 3, and Compound 4.

2. The composition of claim 1, wherein the siRNA has a sequence comprising SEQ ID NO: 1, or a sequence having at least 80% identity to SEQ ID NO: 1.

3. The composition of claim 1, further comprising a plurality of tumor-targeted antibodies.

4. The composition of claim 3, wherein the tumor-targeted antibodies are anti-TROP2 antibodies.

5. The composition of claim 1, further comprising one or more imaging agents, radioimaging agents, or radiotherapeutic agents.

6. (canceled)

7. (canceled)

8. (canceled)

9. (canceled)

10. A pharmaceutical composition comprising the composition of claim 1, and a pharmaceutically acceptable carrier, optionally wherein the composition is formulated as a liquid suitable for injection.

11. (canceled)

12. A method of treating cancer in a subject in need thereof, the method comprising administering to the subject the pharmaceutical composition of claim 10, optionally wherein administration is by an intravenous route or by intratumoral injection.

13. The method of claim 12, wherein the cancer is selected from the group consisting of breast cancer, liver cancer, kidney cancer, lung cancer, ovarian cancer, and bone cancer.

14. The method of claim 12, wherein the cancer is a leukemia or lymphoma.

15. The method of claim 12, wherein the cancer is breast cancer, optionally wherein the breast cancer is refractory to standard therapy or is recurrent.

16. (canceled)

17. (canceled)

18. The method of claim 15, wherein the breast cancer is unresectable locally advanced or metastatic.

19. The method of claim 15, wherein the breast cancer is negative for expression of one or more of estrogen receptor, progesterone receptor, and human epidermal growth factor receptor 2 (HER2).

20. The method of claim 19, wherein the breast cancer is negative for expression of all three of estrogen receptor, progesterone receptor, and human epidermal growth factor receptor 2 (HER2).

21. The method of claim 12, wherein the method further comprises administering an immunotherapy agent to the subject.

22. The method of claim 21, wherein the immunotherapy agent is a programmed cell death protein 1 (PD-1) or programmed cell death ligand-1 (PD-L1) inhibitor.

23. The method of claim 21, wherein the immunotherapy agent is selected from the group consisting of pembrolizumab, nivolumab, cemiplimab, dostarlimab, atezolizumab, avelumab, and durvalumab.

24. (canceled)

25. (canceled)

26. A method of eliciting an anti-tumor immune response in a subject having cancer, the method comprising administering to the subject the pharmaceutical composition of claim 10.

27. A method for clinical imaging of a MYC-expressing tumor in a subject, the method comprising administering to the subject the composition of claim 5.

28. A method of treating a solid tumor in a subject in need thereof, the method comprising administering to the subject the pharmaceutical composition of claim 10, wherein the tumor cells express high levels of MYC or have high levels of MYC activity compared to non-tumor cells of the same tissue type.


SEQUENCE LISTING (3 sequences)

SEQ ID NO: 1 - RNA, 19 nt, synthetic construct
  source 1..19: mol_type = other RNA
cgatgttgtttctgtggaa

SEQ ID NO: 2 - RNA, 19 nt, synthetic construct
  source 1..19: mol_type = other RNA
gaaacgacgagaacagttg

SEQ ID NO: 3 - RNA, 19 nt, synthetic construct
  source 1..19: mol_type = other RNA
ccactcaccagcacaacta